(19) United States
(12) Patent Application Publication
(10) Pub. No.: US 2015/0087594 A1
(43) Pub. Date: Mar. 26, 2015

(54) INDUCED NEURAL STEM CELLS

(71) Applicant: MERCK PATENT GMBH, Darmstadt (DE)

(72) Inventors: Frank Oliver Stefan Edenhoffer, Erfstadt (DE); Philipp Woersdoerfer, Bonn (DE); Yenal Bernhard Lakes, Bonn (DE); Marc-Christian THIER, Bad-Muenstereifel (DE); Oliver Bruestle, Bonn (DE)

(21) Appl. No.: 14/387,126

(22) PCT Filed: Feb. 27, 2013
(86) PCT No.: PCT/EP2013/000573

§ 371 Date: Sep. 22, 2014

(30) Foreign Application Priority Data
Mar. 21, 2012 (EP) 12001981.5

(51) Int. Cl. C12N 5/0797; C12N 5/0793; C12N 5/079

(52) U.S. Cl. CPC C12N 5/0623; C12N 5/0622; C12N 5/0619; C12N 2506/13; C12N 2506/08; C12N 2501/603
USPC 514/17.7; 435/377; 435/366; 435/325

(57) ABSTRACT

The present invention relates to a method of deriving an induced neural stem cell (iNCS) by nuclear reprogramming of a somatic cell, wherein the method comprises a step of contacting the somatic cell with Oct4 protein or a functionally equivalent analogue, variant or fragment thereof for a limited time period, as well as an induced neural stem cell obtained by this method.

The present invention relates to a method of deriving an induced neural stem cell (iNSC) by nuclear reprogramming of a somatic cell, wherein the method comprises a step of contacting the somatic cell with an Oct4 protein or a functionally equivalent analogue, variant or fragment thereof, for a limited time period, as well as an induced neural stem cell obtained by this method.

Induced pluripotent stem (iPS) cells can be obtained by reprogramming somatic cells using the reprogramming factors Oct4, Klf4, cMyc and Sox2 (EP 1970446 A1). These patient-specific cells are expected to be of use in cell transplantation therapies for the treatment of various diseases. Although substantial progress has been made in terms of improving the efficiency and robustness of reprogramming, the derivation of patient-specific iPS cells still is a lengthy and cumbersome procedure. Moreover, disease-related applications require subsequent re-differentiation into the desired cell type and incomplete differentiation of iPS cells carries the risk of tumor induction by remaining undifferentiated iPS cells.

As an alternative, factor-driven reprogramming of fibroblasts has been reported to directly yield other somatic cell types such as neurons (e.g. Vierbuchen et al. 2010, Nature 463, 1035-1041), cardiomyocytes (e.g. Ieda et al. 2010, Cell 142, 375-386), hepatocyte-like cells (Huang et al. 2011, Nature 475, 386-389), as well as blood (Szabo et al. 2010, Nature 468, 521-526) and neural progenitors (NPCs) (Kim et al. 2011, PNAS 108, 7838-7843; Lujan et al. 2012, PNAS 109, 2527-2532).

This transdifferentiation from one differentiated cell type into another can be achieved through overexpression of transcription factors. However, these transdifferentiation protocols result in somatic cell populations with little or no proliferation potential. Moreover, transdifferentiated cells exhibit limited differentiation capability. For example, Kim et al. (see above) discloses that differentiation of transdifferentiated NPCs yields neurons and astrocytes, whereas the oligodendrocyte differentiation capability remaines unclear. Moreover, the NPCs are reported to lose their ability to form colonies within 3-5 passages.

Although neural stem cells (NSCs) can be derived either from somatic tissue or pluripotent sources (e.g. Conti et al. 2005, PLoS Biol 3, e283; Tropepe et al. 2001, Neuron 30, 65-78), the artificial induction of stably expandable NSCs cells has remained elusive.

The object of the present invention was therefore to provide a method of deriving induced neural stem cells (iNSC) being fully expandable and able to differentiate into multiple lineages in vitro and in vivo from somatic cells.

Surprisingly, it has now been found that the direct derivation of stably expandable NSCs from somatic cells can be effected by a modification of the iPS protocol in a way that somatic cells are contacted with an Oct4 protein or a functionally equivalent analogue, variant or fragment thereof for a limited time period.

Therefore, in a first aspect, the present invention is directed to a method of deriving an induced neural stem cell (iNCS) by nuclear reprogramming of a somatic cell, wherein the method comprises a step of contacting the somatic cell with an Oct4 protein or a functionally equivalent analogue, variant or fragment thereof for a limited time period.

A key aspect of the present invention is the contacting of the somatic cell with an Oct4 protein or a functionally equivalent analogue, variant or fragment thereof.

The Oct4 (octamer-binding transcription factor 4) protein'(also: POU5F1) belongs to the POU family of transcription factors and is a homeodomain-containing transcription factor that has been shown to be critical in the induction and maintenance of the pluripotent stem cell state. Down regulation of Oct4 expression in embryonic stem cells causes them to differentiate and lose their pluripotency. The Oct4 gene is localised on human chromosome 6p21.31 and the nucleotide sequence of the gene has been reported by Scholer et al (1989, EMBO J, 8 (9), 2543-2550).

According to the present invention the phrase “functionally equivalent” is intended to mean that the variant, analogue or fragment is also effective in inducing the formation of iNSCs in the somatic cells treated according to the method of the present invention and preferably a given quantity of the analogue, variant or fragment is at least 10%, preferably at least 30%, more preferably at least 50, 80, 90, 95 or 99% as effective as an equivalent amount of Oct4 or the transcription factor from which the analogue, variant or fragment is derived. Determination of the relative efficacy of the analogue, variant or fragment can be carried out by utilizing a prescribed amount of the analogue, variant or fragment in the method according to the present invention and then comparing the iNSCs achieved against the same amount of Oct4 protein or transcription factor from which the analogue, fragment or variant is derived.

Analogues are intended to encompass other POU transcription factors than Oct4 such as Brn1, Brn2 (He et al., 1989, Nature 340(6228):35-41), and Brn4 (Douville et al., Douville et al., Mamm Genome, 5(3):180-2, 1994) or neurogenic transcription factors, such as TLX (Jackson et al., Genomics 50 (1): 34-43, 1998), Bmi1 (Alkema et al., Hum. Mol. Genet. 2 (10): 1597-603, 1993), Hes5 (Akazawa et al., J Biol Chem. 267(30):21879-85, 1992), Brn1 and Brn2 (He et al., Nature 340(6228):35-41., 1989), Brn4(Douville et al., Mamm Genome, 5(3):180-2.1994), Pax6(Glaser et al., Nat Genet. (3):232-9, 1992), Sox1(Malas et al., Mamm Genome. 8(11):866-8, 1997), Sox3(Stevanovic et al., Hum Mol Genet. 2(12):2013-8, 1993), Sox10 (Southard-Smith et al., Nat Genet. 18(1):60-4 1998), PLZF(Avantaggiato et al., J Neurosci. 15(7 Pt 1):4927-42 1995), Hes1(Thomas et al., Nucleic Acids Res., 20(21):5840, 1992), Dach1 (Davis et al., Dev Genes Evol. 209:526-536, 1999), DIx1 (Price et al., Nature (London) 351, 748-751, 1991), Gli1, Gli2, Gli3 (Hui et al., Dev Biol. 162(2):402-13., 1994), 102 (Neuman et al., Dev Biol. 160(1):186-95, 1993), ID4 (Riechmann et al., Nucleic Acids Res. 11; 22(5):749-55, 1994), Olig2 (Lu et al., Neuron. 25(2):317-29, 2000, Zhou et al., Neuron, 25(2):331-43, 2000) . . . . These factors are known from literature and can be obtained by methods known to those skilled in the art.

Variants are intended to encompass proteins having amino acid sequences differing from the protein from which they are derived by virtue of the addition, deletion or substitution of one or more amino acids to result in an amino acid sequence that is preferably at least 60%, more preferably at least 80%, particularly preferably at least 85, 90, 95, 98, 99 or 99.9% identical to the amino acid sequence of the original protein. The variants specifically include polymorphic variants and interspecies homologues.

The term “fragments” encompasses fragments of a protein that are of at least 10, preferably at least 20, more preferably at least 30, 40 or 50 amino acids in length and which are functionally equivalent to the protein of which they are fragment.

Preferably, Oct4 protein is used in the method according to the present invention.

The method comprises a step of contacting the somatic cell with an Oct4 protein or a functionally equivalent analogue, variant or fragment thereof for a limited time period. The limited time period is crucial for the method of the present invention.

According to the present invention “limited time period” means that the exposure of the somatic cell to the influence of the Oct4 protein or a functionally equivalent analogue, variant or fragment thereof is restricted to a certain period of time which is shorter than the entire time period needed for reprogramming towards pluripotency.

In case of an functionally equivalent analogue, variant or fragment of Oct4 it can also be preferred if the limited period of time is extended to the entire time period needed for reprogramming towards pluripotency. This option is suitable, if the functionally equivalent analogue, variant or fragment shows limited functionality compared to Oct4.

In other cases, the limited time period is preferably 10 days or less, more preferably 2 to 5 days, most preferably 3 to 5 days.

Preferably, the step of contacting the somatic cell with the Oct4 protein or a functionally equivalent analogue, variant or fragment thereof, is effectuated at the beginning of the method, i.e. during the initial phase of reprogramming. That means that the limited time period of contacting the somatic cell with the Oct4 protein or a functionally equivalent analogue, variant or fragment thereof, lies preferably within the first 5 days of the method.

It is important to observe the limited time period since it ensures that the somatic cell develops into an induced neural stem cell. Longer or not strongly regulated time periods of contacting the somatic cells with the Oct4 protein favours the formation of induced pluripotent stem cells (iPSC).

Therefore it is important that the step of contacting the somatic cell with the Oct4 protein or a functionally equivalent analogue, variant or fragment thereof, is strongly regulated. This can be effectuated by a variety of different means:

According to one embodiment of the present invention this can be effectuated by delivery of cell-permeable Oct4 protein or its functionally equivalent analogue, variant or fragment.

The delivery of Oct4 protein into the cell can be achieved by techniques well-known in the art, as utilising detergent, bacterial toxin or electroporation, permeabilisation, liposomal delivery or with the use of cell-permeant peptide vectors or polyethylene glycol (PEG).

Preferably, a cell-permeant Oct4 protein is used which can enter the target cell without requiring addition of cell permeabilisation agents.

In particular, the use of a cell-permeable Oct4-TAT protein is preferred as disclosed in Thier et al. (2010, Int J Dev Biol 54, 1713-1721) and Bosnali et al. (2008, Biol Chem 389, 851-861).

It is preferred that the Oct4 protein or analogue or variant thereof utilized in the above described techniques is at least to some extend isolated and purified from other components of a cytoplasmic extract from which it may be obtained.

The Oct4 protein or its functionally equivalent analogues or variants may be produced recombinantely or may be isolated from mammalian cells.

For example, a recombinant Oct4 protein may be obtained via secretion by cells producing Oct4 after appropriate modification, for example by introducing a secretory signal into the sequence, or may be isolated from bacteria transfected with an Oct4 construct.

Throughout the specification the terms “isolated” and “purified” are intended to define that the substance is at least 50% by weight free from proteins, antibodies and naturally-occurring organic molecules with which it is endogenously associated. Preferably, the proteins are at least 75% and more preferably at least 90%, 95% or 99% by weight pure. A substantially pure substance may be obtained by chemical synthesis, separation of the protein from natural sources or production of the protein in a recombinant host cell that does not naturally produce the protein. Proteins may be purified using standard techniques. The purity can be measured using polyacrylamide gel electrophoresis, column chromatography, optical density, HPLC analysis or western blotting. Preferred methods of purification include immuno precipitation, column chromatography such as immuno affinity chromatography, magnetic bead immuno affinity chromatography and panning with a plate-bound antibody. In the case where the Oct4 protein is produced by recombinant technology, the protein may be purified by virtue of specific sequences incorporated into the protein, as, for example, through Nickel column affinity where the protein has 6 or more histidine amino acids incorporated into the sequence.

The contacting of the cells with cell-permeable Oct4 protein can for example be achieved by culturing the cells in a medium supplemented with the protein. Within this regard it is important that the protein is only added to the media during the limited time period.

The amount of Oct4 protein or its analogues, variants or fragments contacted with the somatic cells which is effective in order to derive iNSCs, can readily be optimised by a person skilled in the art. The effective amount may vary depending upon the technique adopted for contacting the somatic cells with Oct4 protein and may also depend upon the types and species of cell utilised, cell culture conditions and whether the method is conducted in vivo or in vitro. Typically, the amounts of Oct4 protein or functionally equivalent analogue, variant or fragment, thereof fall within the range of 0.01-12 μg/ml per 10⁵ target cells.

According to another embodiment of the present invention the step of contacting the somatic cell with the Oct4 protein or a functionally equivalent analogue, variant or fragment thereof, is effectuated by delivery of mRNA encoding for Oct4 protein or its functionally equivalent analogue, variant or fragment. The mRNA is a non-integrating RNA, i.e. it does not integrate into the genome of the target cell.

For this purpose, the respective mRNA can be purified from cells after lysis or synthesized chemically or enzymatically from a DNA template using an RNA polymerase, as for example disclosed in Warren et al (2010, Cell Stem Cell 7, 618-630). For example, mRNA can be synthesized using the Ampliscribe™T7-Flash™ Transcription Kit (Epicentre, Illumina company, Madison, Wis.).

The purified mRNA can then be delivered into the cell by a variety of means such as microinjection, electroporation or lipid-mediated transfection. As an example, the mRNA transfection can be performed using the TransIT-mRNA reagent (Mirus Bio, Madison, Wis.).

In order to achieve a constant level of Oct4 protein or its functionally equivalent analogue, variant or fragment during the limited time period, it can be necessary that the somatic cells are transfected more than once, e.g. twice. As an example, the cells might be transfected at day 1 and day 3 of the method according to the present invention.

The amount of mRNA transfected can be determined by a person skilled in the art and depends on the cells and culture conditions employed. For example, the amount of mRNA per transfection step may lie between 0.1 and 8 μg/6-well with approximately 100,000-200,000 cells/6-well.

Alternatively, in a further embodiment of the present invention the step of contacting the somatic cell with the Oct4 protein or a functionally equivalent analogue, variant or fragment thereof, is effectuated by doxycycline-induced expression of the respective protein, as disclosed by Soldner et al. (2009, Cell 136, 964-977).

The respective genes may be introduced into the somatic cells by using methods commonly used in animal cell transfections. Specific examples are methods using vectors, calcium phosphate, lipofection, electroporation or microinjection. For introduction efficiency, methods using vectors are preferable. The vectors may be, for example, virus vectors, non-virus vectors, or artificial viruses. Considering safety, non-integrating viral vectors such as Sendai virus (Fusaki et al. 2009, Proc. Jpn. Acad. Ser. B, Phys. Biol. Sci., 85, 348-362) or deletable vectors such as Cre-excisable vectors (Sommer et al. 2009, Stem Cells 28, 64-74) are preferably used. Therefore another preferred method of contacting the somatic cell with the Oct4 protein or a functionally equivalent analogue, variant or fragment thereof is by infection employing non-integrating viruses carrying the respective genes.

In this embodiment a lentiviral vector encoding for the Oct4 protein is used, wherein the expression of the Oct4 protein is inducible and thus controllable by doxycycline (dox).

Lenti-viruses comprising the respective plasmide encoding for doxycycline-inducible Oct4 expression can be produced by common methods, such as using the 293FT packaging cell line (Life Technologies, Carlsbad, Calif.). The conditions for infection with the lentiviruses can easily be determined by a person skilled in the art.

After transfection of the somatic cells the presence of doxycycline during the limited time period results in the temporarily expression of Oct4. Doxycycline can for example be delivered to the cells by using doxycycline containing media.

Alternatively, all other possible methods may be used, including introducing an expression vector for Oct4 protein into supporting cells and using those transfected cells as co-culture cells, and using a culture supernatant or other cell product of those transfected cells.

Preferably, Oct4 protein transduction or mRNA transfection is employed. These methods are preferred since they allow a more precise and unambiguous control over Oct4 activity in order to be able to strictly control the limited time period.

The somatic cell may be selected from hepatocytes, fibroblasts, endothelial cells, B cells, T cells, dendritic cells, keratinocytes, adipose cells, epithelial cells, epidermal cells, chondrocytes, cumulus cells, neural cells, glial cells, astrocytes, cardiac cells, oesophageal cells, skeletal muscle cells, skeletal muscle cells, skeletal muscle satellite melanocytes, hematopoietic cells, osteocytes, adipocytes, cord-blood cells, dental cells, macrophages, monocytes or mononuclear cells.

Preferably, the employed somatic cells are fibroblasts or keratinocytes.

Preferably, a mammalian cell is used as somatic cell.

The cells utilised according to the present invention may be derived from any of a variety of mammalian organisms, including, but not limited to humans, primates, such as chimpanzees, gorillas, baboons, organutans, laboratory animals such as mice, rats, guinea pigs, rabbits, domestic animals, such as cats and dogs, farm animals, such as horses, cattle, sheep, goats or pigs or captive wild animals such as lions, tigers, elephants, buffalo, deer or the like. In a treatment method it is preferable that the used cells in treating a particular mammalian patient are derived from an individual of the same species. Most preferably, in order to minimise problems associated with immune rejection, cells used to treat a particular patient will be derived from the same patient.

Preferably, the somatic cell is a human cell.

The method according to the present invention may be conducted in vivo within a mammalian organism or may be conducted in vitro employing mammalian cells.

By the phrase “deriving an induced neural stem cell (iNCS) by nuclear reprogramming of a somatic cell” it is meant that as a result of the method at least some, preferably at least 0.01%, more preferably at least 1%, particularly preferably at least 10% and most preferably at least 40% of the somatic cells treated according to the method of the present invention will demonstrate features of neural stem cells as a result of the treatment according to the method of the present invention. The iNCS will be expandable for more than 10 passages, preferably for more than 20 passages, particularly preferable for more than 30 passages, most preferably for more than 50 passages.

The presence of induced neural stem cells (iNCS) may for example be detected by RT-PCR analyses, immunofluorescence stainings as well as microarray analyses (see Example 2).

According to the present invention an induced neural stem cell (iNCS) is a cell artificially derived from a somatic cell exhibiting a high degree of similarity with a natural neural stem cell compared to a somatic cell, preferably a similarity of at least 20%, particularly preferably of at least 40%, and most preferably of at least 60%. The similarity can, for example, be determined by the activity of specific genes, such as Foxg1, Nes, Bmi1 and Olig2, which are strongly up-regulated in iNSCs and NSCs compared to somatic cells, or target cell-specific genes (i.e. for fibroblasts Col1a1, Col3a1, Dkk3 or Thy1), which are downregulated in iNSCs and NSCs compared to somatic cells.

A further aspect of the present invention is that the induced neural stem cell is derived directly from the somatic cell, without passing through a pluripotent stage.

The somatic cell may also be contacted with one or more other transcripition factors or their functionally equivalent analogues, variants or fragments. Such transcripition factors may be selected from Sox2, Klf4, and cMyc. These factors may be contacted with the cell for example as cell-permeable proteins or may be introduced into the cells by transfection of the gene encoding these transcription factors (as DNA or RNA) or by viral transduction.

Therefore, a further embodiment of the present invention is a method as described above, characterized in that the method further comprises contacting the somatic cell with transcription factors selected from Sox2, cMyc and/ or Klf4 or their functionally equivalent analogues, variants or fragments.

The transcriptional factors or their functionally equivalent analogues or variants may be produced recombinantely or may be isolated from mammalian cells. Concerning their production and introduction into the cell it is referred to the techniques described above for the introduction of the Oct4 protein.

In a further embodiment of the present invention, the transcription factors may be delivered by introducing mRNA encoding for Klf-4, cMyc, and Sox-2 into the cell. Concerning the production and introduction of the mRNA into the cell it is referred to the techniques described above for the introduction of the Oct4 mRNA.

Alternatively, these transcription factors may be introduced into the cell by transfection or viral transduction of the DNA or RNA encoding the respective factors.

The respective genes may be introduced into the somatic cells by using methods commonly used in animal cell transfections and as already described above. Virus vectors such as Sendai virus and Cre-deletable viruses are preferably used. When vectors are used, the genes encoding for the different transcription factors may be incorporated into different vectors, or may be incorporated in the same single vector.

Preferably, the somatic cell is contacted with the transcription factors by infection with retroviruses encoding for the transcripition factors (Takahashi and Yamanaka 2006, Cell 126, 663-676). Suitable plasmids can for example be obtained from Addgene (Cambridge, Mass.): pMXs-Sox2 (Addgene plasmid 13367), pMXs-c-Myc (Addgene plasmid 13375) and pMXs-K1f4 (Addgene plasmid 13370).

Retroviruses can be generated by common methods, e.g. by using the Platinum E and A, respectively, retroviral packaging cell line (Cell Biolabs, Inc, San Diego, Calif.). Suitable conditions for infecting the cells with the retroviruses can easily be determined by a person skilled in the art.

A preferred embodiment of the present invention relates to a method as described above, wherein the somatic cell is constitutively contacted with the transcription factors Sox2, cMyc and Klf4 or their functionally equivalent analogues, variants or fragments.

Constitutively means that the cells are contacted with the respective factors during the entire process of reprogramming, which is a longer period of time than the limited time period of contacting the cell with the Oct4 protein.

This can for example be effectuated with the aid of the above described retroviruses which allow for the integration of the viral RNA after transcription into DNA into the genome of the somatic cell.

A further preferred embodiment of the present invention relates to a method as described above, wherein the somatic cell is contacted with one, or two, ore three, or more additional transcription factors, such as TLX, Bmi1, Hes5, Brn1, Brn2, Brn4, Pax6, Sox1, Sox3, Sox10, PLZF, Hes1, Dach1, Dlx1, Gli1, Gli2, Gli3, ID2, ID4, Olig2 or their functionally equivalent analogues, variants or fragments. Preferably these additional factors are also applied during a limited period of time, as described above. In another embodiment of the invention these factors are applied constitutively.

The method according to the present invention allows for generating neurosphere-like colonies of stably expandable induced NSC (iNSC) lines maintaining their tripotent developmental potential over prolonged expansion (more than 50 passages) and being not dependent on sustained expression of reprogramming factors. These induced neural stem cells (iNSCs) uniformly display morphological and molecular features of NSCs such as the expression of Nestin, Pax6, and Olig2 and have a similar genome-wide transcriptional profile to brain-derived NSCs.

The uniformity and stability of growth characteristics and marker expression together with long-term maintenance of tripotential neural differentiation capability show that iNSCs represent a stably proliferating somatic stem cell type.

In a further aspect the present invention also relates to an induced neural stem cell obtained by a method as described above.

Moreover, iNSCs can differentiate into all three main neural lineages, i.e. into neurons, astrocytes and oligodendrocytes.

Therefore, a method as defined above, characterized in that the induced neural stem cell further differentiates into a neuron, an astrocyte or an oligodendrocyte, is a further aspect of the present invention.

Another aspect of the present invention is also a neuron, an astrocyte or an oligodendrocyte obtained from an induced neural stem cell obtained by a method as described above.

Any method suited for inducing differentiation into neurons, astrocytes or oligodendrocytes can be used as the culture method for preparing these cells from iNSCs. The person skilled in the art can easily determine the necessary culture conditions (see below). A typical protocol for deriving these cells can for example be found in Glaser et al. (2007, PLoS One 2, e298). As a specific example, iNSCs can be grown in 10% FCS-containing medium supplemented with NEAA and glutamine in order to derive astrocytes.

Alternatively, the differentiation into neurons can be achieved by plating iNSCs onto POL-coated dishes in a mix of Neurobasal Medium and DMEM/F12 supplemented with N2, B27, BDNF and ascorbic acid. To derive oligodendrocytes, iNCS can be cultivated in DMEM/F12 with N2, PDGF and Forskolin, whereas PDGF and Forskolin are later replaced by 3,3,5-triiodothyronine (T3) hormone and ascorbic acid.

iNSCs may provide a safe and robust cellular platform for the generation of patient-specific neural cells for biomedical applications.

Therefore, another embodiment of the present invention is a method of treatment and/or prophylaxis of a degenerative disease or injury in a mammal, which comprises removing from the mammal one or more responsive somatic cells and culturing the cells in a suitable medium, contacting the cells with an Oct4 protein or a functionally equivalent analogue, variant or fragment thereof for a limited time period, and subsequently returning the cells to the patient.

An alternative hereto is also a method of treatment and/or prophylaxis of a degenerative disease or injury in a mammal, which comprises contacting responsive somatic cells of the patient with an Oct4 protein or a functionally equivalent analogue, variant or fragment thereof for a limited time period.

As mentioned above, the method according to the present invention may be conducted in vivo or in vitro. By in vivo treatment it is intended to mean that the method is conducted upon the somatic cells while they are located within the organism concerned. In vitro application means that mammalian cells are exposed to the method according to the invention in an in vitro cell culture setting. After exposure of the cells to the method, the treated cells transformed into iNSCs may be further treated to obtain the desired lineages as neurons, astrocytes or oligodendrocytes.

This invention relies upon routine techniques in the filed of cell culture. Suitable techniques can easily be determined by a person skilled in the art using known methodology. In general, the cell culture environment includes consideration of such factors as the subtrate for cell growth, cell density and cell contact, the gas phase, the medium and temperature.

Generally, the cells can be grown in suspension or under adherent conditions.

In a preferred embodiment, the cells are grown under adherent conditions. Typically, plastic dishes, flasks, roller bottles or microcarriers in suspension are used. Other artificial substrates can be used such as glass or metals. The substrate may be treated by etching or by coating with substances such as collagen, chondronectin, fibroncetin, and laminin. The type of culture vessel depends on the culture conditions, e.g. multi-well plates, Petri dishes, tissue culture tubes, flasks, roller bottles and the like. Alternatively, the cells may be grown in suspension as three dimensional aggregates. Suspension cultures can be achieved by using, e.g. a flask with a magnetic stirrer or a large surface area paddle, or on a plate that has been coated to prevent the cells from adhering to the bottom of the dish.

Cells are grown at optimal densities that are determined empirically based on the cell type. For example, a typical cell density for Oct4-GiP mouse embryonic fibroblast (MEFs) cultures varies from 100,000 to 600,000 cells per well of a 6-well dish. Cells are passaged when the cell density is above optimal.

Cultured cells are normally grown in an incubator that provides a suitable temperature, e.g. the body temperature of the animal from which the cells were obtained. Generally, 37° C. is the preferred temperature for cell culture. Most incubators are humidified to approximately atmospheric conditions.

Important constituents of the gas phase are oxygen and carbon dioxide. Typically, atmospheric oxygen tensions (20%) are used for cell cultures, though for some cell types lower oxygen concentrations of 10%, 5% or 2% are preferred. Culture vessels are usually vented into the incubator atmosphere to allow gas exchange by using gas permeable caps or by preventing sealing of the culture vessels. Carbon dioxide plays a role in pH stabilisation, along with buffer in the cell media and is typically present at a concentration of 1-10% in the incubator. The preferred CO₂ concentration is typically 5%.

Defined cell media are available as packaged, premixed powders or presterilised solutions. Examples of commonly used media include DMEM (Dulbecco's modified eagle's medium), RPMI 1640, Iscove's complete media, or McCoy's Medium. Typically, DMEM is used in the methods of the invention. Defined cell culture media are often supplemented with 5-20% serum, typically heat inactivated, e.g. human, horse, calf, and fetal bovine serum. Lower concentrations are also possible, if the reduced serum content is compensated by application of serum replacement. Typically, in the present invention, media are supplemented with 2% FCS (fetal calf serum) and 8% serum replacement, because, although FCS is required for efficient propagation of fibroblasts, it may induce unwanted terminal differentiation of NSCs.

The culture medium is usually buffered to maintain the cells at a pH preferably from 7.2-7.4.

Other possible supplements to the media include, e.g., antibodies, amino acids, sugars or growth factors such as growth factors or growth promoting agents suitable for the maintenance of pluripoteny. Examples are hepatocyte growth/factor (HGF), Insulin-like growth factor-1 (IGF-1), members of the fibroblast growth factor (FGF) family, members of the bone morphogenic protein (BMP) family, and epidermal growth factor (EGF).

### DESCRIPTION OF THE FIGURES

FIG. 1. Generation and characterization of induced neural stem cells (iNSCs) from mouse embryonic fibroblasts:

Scale Bars: 50 μm.

(A) Schematic drawing of the experimental setup and strategy to derive iNSCs.

(B) Neurosphere-like colony at day 25 with axonal structures processing out of the sphere.

(C) Neurosphere-like colony at day 27 stained with a specific antibody directed against the neuronal marker 6-III-tubulin.

(D) Neurosphere-like colony isolated by manual picking, grown on a POL-coated culture dish. iNSCs are migrating out of the sphere (defined as passage 0).

(E) iNSCs (passage 1) are dissociated to a single cell suspension, seeded onto a POL-coated culture dish and cultured for 2 days.

(F-I) Immunofluorescence analysis of neural stem cell marker proteins in iNSCs using specific antibodies directed to Nestin and Olig2 (G), Sox2 and BLBP (H) as well as Vimentin (I). A brightfield micrograph is shown in F.

(J-K) Phase contrast pictures of iNSCs in NS propagation medium at passage 5 (J) and 31 (K).

(L-M) iNSCs at passage 12 (L) and passage 31 (M) are stained for neural stem cell markers Nestin and Pax6.

(N-O) iNSCs form secondary neurospheres when kept in suspension culture (N). After plating on POL-coated culture dishes spheres get adherent and iNSCs migrate out (O).

(P) Mean doubling time (mdt) and growth curve of three iNSC lines (Cl. 3-5) in comparison to brain-derived NSCs (NS). Abbr.: sd=standard derivation. N=6.

FIG. 2. Direct conversion of adult mouse TTFs and RT PCR analysis of MEF-derived iNS cells

(A-G) Tail tip fibroblasts (TTFs) were retrovirally transduced with Klf4, Sox2 and c-Myc. At day 1 and 3 after splitting, synthetic Oct4-encoding mRNA was transfected. Beginning on day 15 p.i., neurosphere-like structures appeared in the culture dish, which were isolated on day 21. (A) Neurosphere-like structure on primary plate at day 19. (B) Isolated colony showing characteristic outgrowth. (C) Immunostaining of SKC-transduced control cells for Pax6 and β-III-tubulin on day 15 p.i. (D-G) Cells additionally transfected with Oct4 form colonies that stain positive for Pax6 and β-III-tubulin exhibiting NSC-like morphology (D). Pax6 signal is localized in the nucleus, whereas TUJ1 antibody stains non-nuclear compartments (E-G). (H) Reverse transcription PCR (RT-PCR) analysis of four iNSC lines (2-5) employing specific primer pairs for the detection of neural stem cell markers Pax6, Blbp, and Sox2. Oct4—(pluripotency marker) and GAPDH-specific primers were used as control. RNA preparations of NSCs, ESCs and mouse embryonic fibroblasts (M) served as controls.

Scale bars: 50 μm

FIG. 3. Analysis of residual fibroblast genes and regional identity of INS cell

(A) Heatmap presentation of residual activity of fibroblast genes in iNSCs. Plotted are differential score values derived from differential gene expression analysis against the MEF signals. The heatmap presents 48 genes (of a total of 30,854 genes) that show a comparable expression level in three iNSC lines (2, 3 ,5) and in MEFs, but have an at least 5-fold higher or lower expression level in the brain-derived control NSCs. (B) Transcriptomal profiling of region-specific markers on three iNSC lines, MEFs as well as primary brain-derived control NSCs. (C) RT-PCR analyses of selected genes employing RNA from iNSC lines. RNA from fetal brain, adult brain and MEFs serve as controls. (D) Primers used for RT-PCRs shown in (B).

FIG. 4. Genome-wide transcriptional profiling of iNSCs and analysis of transgene silencing

(A-D) Homogeneity of gene expression visualized by scatter plot presentation. Shown are plots of the averaged intensities of each group against the induced NSC clone 2 (iNSC2). Detection p-values are less or equal 0.01.

(E-F) Hierarchical cluster analysis and heatmap presentation of microarray expression analysis of MEF cells, three iNS lines (iNSC2, 3, 5), NS control cells (NSC) as well as ES cells (ESC). All samples are processed in at least triplicates to reduce signals arising from processing artifacts. Plotted are the differential score values of 147 genes selected by text-mining for the term ‘neuro’ in the gene definition data provided by GenomeStudio (E) and a subset of selected neural, pluripotency and fibroblast-specific genes (F) as computed by GenomeStudio's differential gene analysis algorithm.

(G) Reverse transcription PCR (RT-PCR) analyses of four iNSC lines (2-5) for sustained expression of the retroviral transgenes as well as endogenous expression of Oct4, Sox2 and Nanog. RNA preparations of uninfected MEFs, SKC-infected MEFs (Tg MEF), ES cells (ESC) as well as a brain-derived NSC line serve as controls. All four iNS lines analyzed exhibit expression of endogenous Sox2 like the NSC samples. Transcription of transgenic factors is not observed. Primer pairs detecting the Glyceraldehyde 3-phosphate dehydrogenase (GAPDH) are used as loading control.

(H) Genomic PCR to verify integration of retroviral constructs used for partial reprogramming. Primer pair detecting endogenous g-actin serves as loading control.

FIG. 5. In vitro-differentiation potential of iNSCs

(A-C) iNSCs differentiate into astrocytes, oligodendrocytes and neurons in vitro as judged by immunofluorescence analyses using specific antibodies directed against GFAP, O4, β-III-tubulin and MAP2ab.

(D-E) iNSC-derived neurons stain positive for the neuron specific marker NeuN. E shows a magnification of D.

(F, H) iNSC-derived neurons expressing mature neuronal marker MAP2ab and GABA, respectively.

(G) Quantification of the differentiation potential of iNSCs into neurons, astrocytes and oligodendrocytes. About 30% of the iNSCs-derived progeny stain TUJ1-positive when cultured in N2 Medium without EGF and bFGF. In serum-containing medium, almost 100% of the cells differentiate into astrocytes. About 3% oligodendrocytes are found after culture in N2 medium with PDGF, Forskolin and T3 followed by N2 medium with ascorbic acid

(I) Staining of iNSC-derived neurons for Synapsin and after three weeks of differentiation.

(J-M) Physiological properties of iNSC-derived neurons assessed by whole-cell patch-clamp recordings. (J) Complex outward current pattern including inactivating and sustained components reminiscent of neurons expressing both A-type and delayed rectifier potassium channels. (K) Current-clamp experiment of an iNS-derived neuron able of firing an action potential, thus demonstrating membrane excitability. (L-M) Local application of either 15 mM L-glutamate (L) or 15 mM GABA (M) elicited a clear current response, indicating the expression of functional neurotransmitter receptors (n=5 and 3, respectively). Stainings are performed after two weeks of differentiation if not otherwise stated. Recordings are done after three weeks.

Scale bars: 50 μm.

FIG. 6. Quantification of iNS cell differentiation.

(A) Tripotent differentiation capability of iNSCs compared to brain-derived NSCs. About 30% of the iNSC-derived progeny stain positive for β-III-tubulin when cultured in N2 medium without EGF and bFGF. In serum containing medium almost 100% of the cells differentiate into astrocytes. Few percent of oligodendrocytes are found after culture in N2 medium with PDGF and Forskolin followed by cultivation in N2 medium with ascorbic acid. (B) Comparison of the neurogenic potential of four different iNSC lines and brain-derived control NSCs. The differentiation potential was measured by immunostainings against p-III-tubulin, GFAP and 04. At least 2,000 cells per cell line were counted.

FIG. 7. Transplantation of iNSCs into the brain of neonatal rats

iNSCs are transplanted into the left and right hemispheres of postnatal myelin-deficient rats. Two weeks after transplantation rats are sacrificed and brain slices analyzed.

(A) M2 antibody marks murine iNSC-derived mouse cells integrated into the rat brain.

(B-D) Immunofluorescence analyses using specific antibodies targeted against astrocyte-specific GFAP (B,C), neuron-specific NeuN (C), and oligodendrocyte-specific PLP (D), M2/GFAP double positive mouse astrocytes are found in close contact with a blood vessel. The white x in (B) indicates the lumen of the vessel. Arrowheads in (C) point to iNSC-derived M2/GFAP double-positive astrocyte processes that surround host neurons.

Scale bars: 50 μm

### EXAMPLES

### Example 1

### Derivation of Neurosphere-Like Clusters by Curtailed Reprogramming

Virus Production

The plasmids pMXs-Sox2 (Addgene plasmid 13367), pMXs-c-Myc (Addgene plasmid 13375) and pMXs-Klf4 (Addgene plasmid 13370) (Takahashi and Yamanaka, 2006, Cell 126, 663-676) are obtained from Addgene (Cambridge, Mass., www.addgene.org). Plasmids encoding FUW-Oct4 and the transactivator m2RTTA are described in Soldner et al. (2009, Cell 136, 964-977). Retroviruses are generated using the Platinum E retroviral packaging cell line (Cell Bioloabs, Inc., San Diego, Calif.), according to the recommendation of the manufacturer. FUW-Oct4 and m2RTTA lenti-viruses are produced using the 293FT packaging cell line (Life technologies, Carlsbad, Calif.) as well as the helper plasmids psPAX2 (Addgene plasmid 12260) and pMD2.G (Addgene plasmid 12259) as described in Brambrink et al. (2008, Cell Stem Cell 2, 151-159).

### Example 1a

### Doxycyline (dox)-Controlled Oct4 Activation

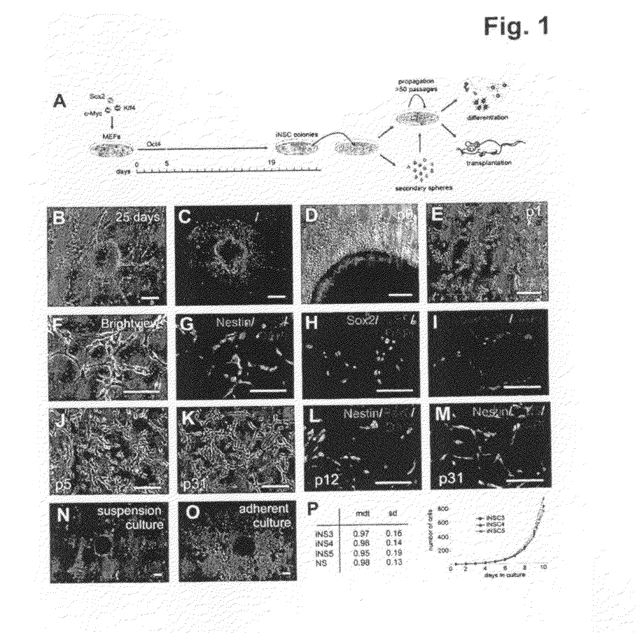

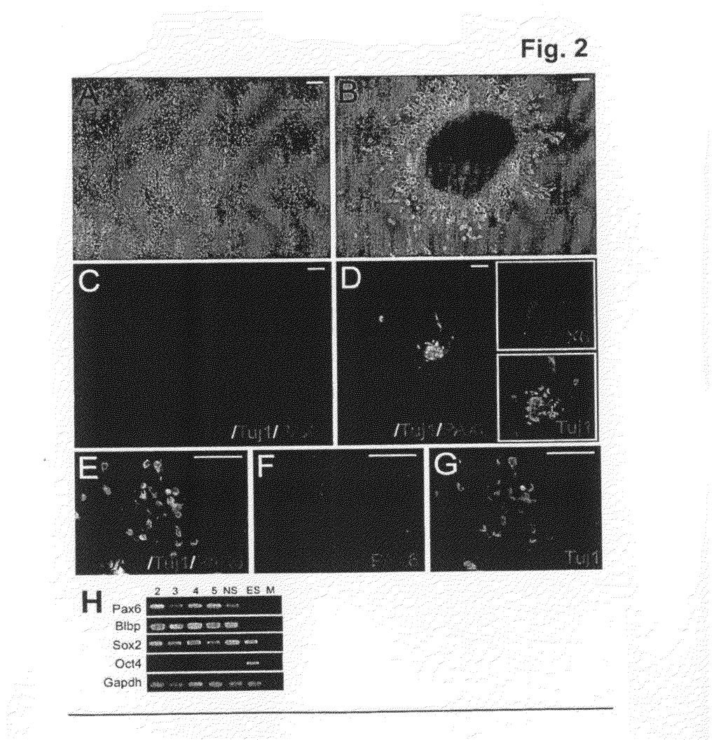

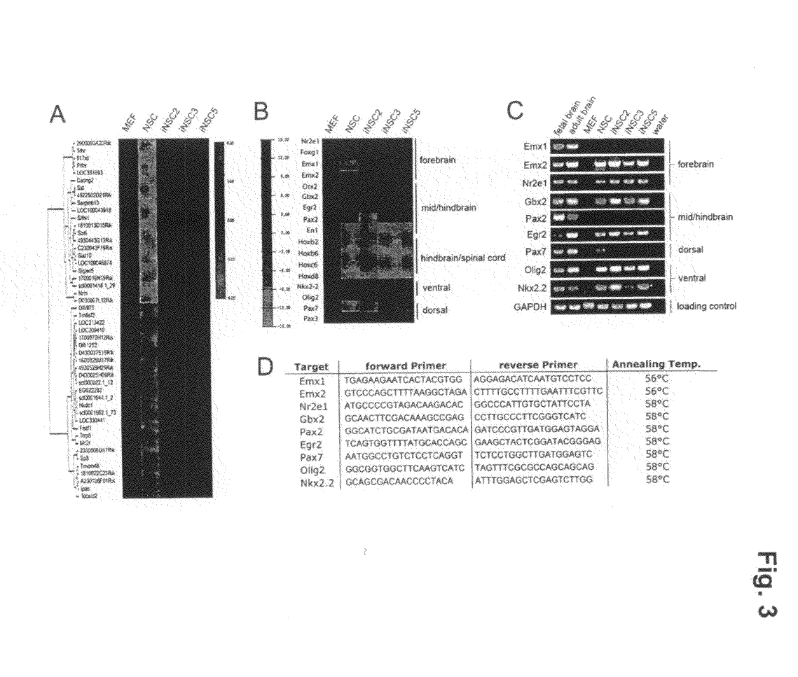

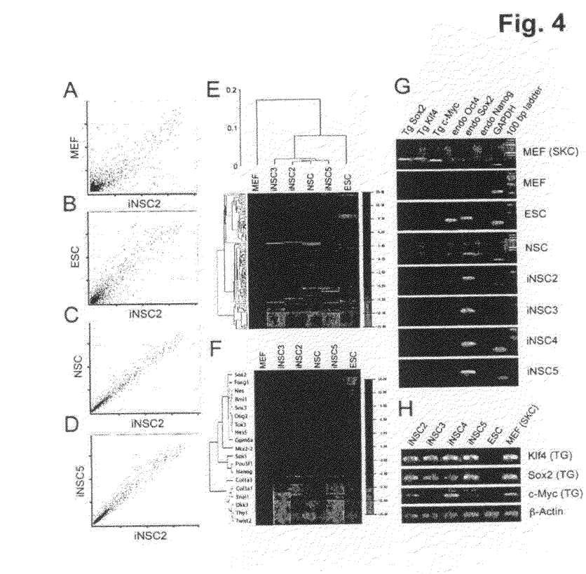

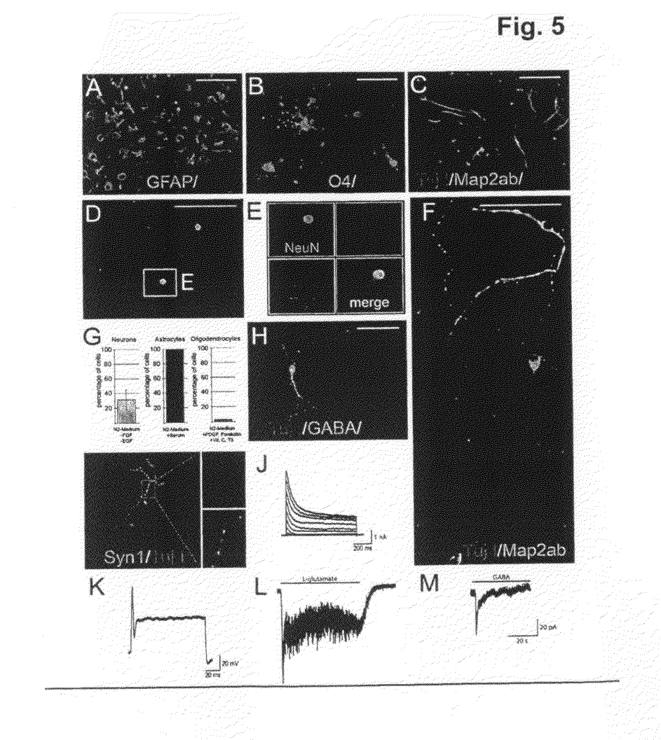

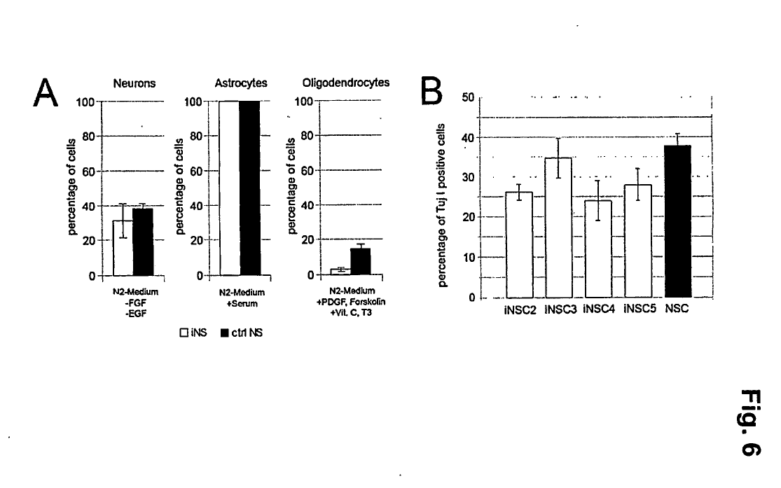

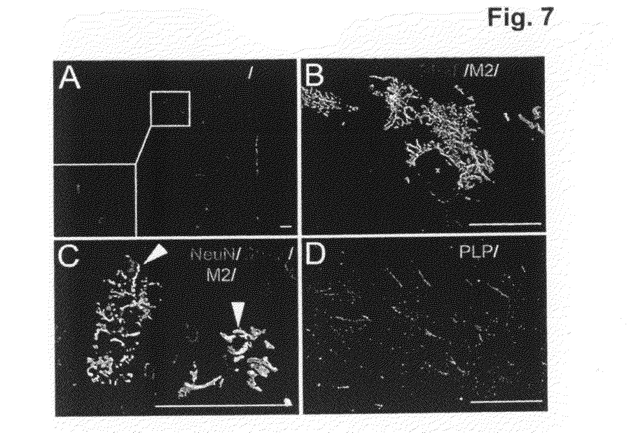

Mouse embryonic fibroblasts (MEFs) are infected with retroviruses constitutively expressing Sox2, Klf4 and c-Myc (SKC) (FIG. 1A).

In order to achieve control of Oct4 activity a lentivirus enabling doxycyline (dox)-controlled Oct4 activation is used (Soldner et al. 2009, Cell 136, 964-977). 130,000 Oct4-GiP reporter MEF cells (Ying et al. 2002, Nature 416, 545-548) are transduced and kept in the presence of doxycycline for up to 5 days to express Oct4 temporarily and yet maintain the expression of the other three factors:

Cell Culture and Infection

For tet-controlled experiments, MEFs are infected with lentiviruses FUW-Oct4 and m2RTTA followed by a second infection with retroviruses encoding for Sox2, Klf4 and c-Myc.

Cells are incubated with viral particles for 16 hours.

1 μg/ml Doxycycline (Sigma, Saint Louis, Mo.) is applied after the virus-containing supernatant is removed. Experiments are carried out in iNS induction medium (DMEM/F12, 2% FCS, 8% Serum Replacement, 1× N2, 2 mM L-glutamine, 1× NEAA and 1000 U/ml ESGRO; all media and cell culture supplements are purchased from Life technologies, Carlsbad, Calif., if not otherwise stated). The medium is changed daily.

Results

Rare neurosphere-like colonies emerge that exhibit neither iPS-like morphology nor GFP fluorescence by eleven days post infection (p.i.). Cultures that are treated with dox for 5 days also yield GFP-positive iPS-like colonies. From this observation it is concluded that a short and strictly regulated pulse of Oct4 expression is critical for NSC induction but prolonged expression of Oct4 favors iPSC induction. Because of the basal activity of the dox-system in the absence of the inducer a tightly regulated abrogation of Oct4 activation sufficient to impede terminal iPS formation is not possible.

### Example 1b

### Direct Delivery of Cell-Permeant Oct4 Protein and mRNA

More precise control over Oct4 activity can be achieved by direct delivery of cell-permeant Oct4 protein (Bosnali and Edenhofer 2008, Biol Chem 389, 851-861; Thier et al. 2010, Int J Dev Biol 54, 1713-1721; Zhou et al. 2009, Cell Stem Cell 4, 381-384) and mRNA (Warren et al. 2010, Cell Stem Cell 7, 618-630), respectively.

Preparation of Oct4-Protein

Protein production is carried out as described in Bosnali and Edenhofer, (2008, Biol Chem 389, 851-861) and Thier et al. (2010, Int J Dev Biol 54, 1713-1721), with modifications: BL21 (DE3) competent bacteria are transformed with the plasmid encoding for Oct4TAT and protein production is induced by addition of Isopropyl-β-D-thiogalactopyranosid (IPTG; Life technologies, Carlsbad, Calif.). The bacteria containing Oct4TAT are collected by centrifugation. For cell lysis, pellets are incubated with disruption buffer (100 mM Tris, 1 mM EDTA, pH 8.0, 3 mM MgCl₂) supplemented with 1 mg/ml lysozyme (Fluka Analytical, St. Gallen, Switzerland) and 10 U/μl benzonase (Novagen/Merck, Darmstadt, Germany). The crude lysate is centrifuged and the pellets containing the inclusion bodies are washed repeatedly with washing buffer (50 mM Tris, 0.1M NaCl, 0.5% Triton-X-100 (Sigma, Saint Louis, Mo.), 0.1% sodium azid, pH 8.0 and 0.1 M Tris, 2 mM EDTA, pH 8.0). The inclusion body fraction is solubilized using 8 M Urea, 50 mM Tris, 1 mM EDTA, 100 mM DTT and dialysed against a buffer comprising 6 M GuaHCL (pH 4.5). In a next step, the denatured protein is refolded by rapid dilution in 50 mM Na₂HPO₄, 1 mM EDTA, 5 mM GSG, 3 M Urea, 20% Glycerol, 1 M L-Arginine and 5% sucrose. The refolded protein is incubated with Ni-NTA agarose beads (Qiagen, Hilden, Germany) and concentrated by affinity chromatography. The protein is eluted with 50 mM Na₂HPO₄, 5 mM Tris, 500 mM NaCl, 250 mM imidazole (pH 7.8). The purity of the Oct4TAT is routinely controlled by SDS-PAGE. For cellular delivery Oct4-TAT protein elution fraction is dialysed against DMEM/F12 (Life technologies, Carlsbad, Calif.) overnight at 4° C. and supplemented with 2% FCS (Life technologies, Carlsbad, Calif.), 8% Serum Replacement (Life technologies, Carlsbad, Calif.), 1× N2 (Life technologies, Carlsbad, Calif.), 2.5% Albumax (200 mg/ml stock, Life technologies, Carlsbad, Calif.), 2 mM L-glutamine (Life technologies, Carlsbad, Calif.), 1×NEAA (Life technologies, Carlsbad, Calif.) and 1000 U/ml ESGRO (mLif)(Life technologies, Carlsbad, Calif.). The medium is pre-conditioned for 1 h at 37° C. and precipitated protein is removed by centrifugation and sterile filtration. Stability and protein concentrations are determined by Western blot or dot blot analysis.

Preparation of Synthetic mRNA Encoding for Oct4

Construction of synthetic mRNA is generated as described in Warren et al. (2010, Cell Stem Cell 7, 618-630) with various modifications: RNA is synthesized using the Ampliscribe™T7-Flash™ Transcription kit (Epicentre, Illumina company, Madison, Wis.) and the capping analogon is directly synthesized using the ScriptCap™ m⁷G Capping System and ScriptCap™ 2′-O-Methytransferase Kit (Cellscript™, Madison, Wis.). All used chemicals are purchased from Carl Roth, Karlsruhe, Germany, if not otherwise specified.

Cell Culture and Infection

For transdifferentiation experiments with recombinant Oct4-TAT protein, Oct4-GiP mouse embryonic fibroblasts (MEFs) are infected with retroviruses encoding for Sox2, Klf4 and c-Myc (see Example 1) (pooled in equal parts) and supplemented with polybrene (4pg/mL; Millipore, Billerica, Mass.).

Cells are incubated with viral particles for 16 hours.

Oct4-TAT protein (Sigma, Saint Louis, Mo.) is applied after the virus-containing supernatant is removed. Experiments are carried out in iNS induction medium (DMEM/F12, 2% FCS, 8% Serum Replacement, 1× N2, 2 mM L-glutamine, 100 μM β-mercaptoethanol, 1× NEAA and 1000 U/ml ESGRO; all media and cell culture supplements are purchased from Life technologies, Carlsbad, Calif., if not otherwise stated). The medium is changed daily.

Direct Conversion of Adult Mouse Tail Tip Fibroblasts (TTFs)

TTFs obtained from WT mice are passaged 2-4 times and retrovirally transfected with Klf4, Sox2 and c-Myc. One day after splitting, synthetic Oct4-encoding mRNA is transfected twice, at day 1 (4 μg/6-well) and day 3 (2.5 μg/6-well) using the TransIT-mRNA reagent (Mirus Bio, Madison, Wis.) according to manufacturers instructions. After 16 hours the medium containing synthetic mRNA and transfection reagent is replaced with fresh medium. From day 1 to day 7 cells are cultured in iNS induction medium, followed by a 1:1 mix of iNS induction and propagation medium from day 8 on. Beginning on day 15, neurosphere-like structures appear in the culture dish, which are subsequently isolated on day 21.

Results

Five days of Oct4 protein transduction into 130,000 SKC-infected fibroblasts generate up to 11 GFP-negative neurosphere-like structures (FIG. 1B). Staining of neurosphere-like colonies at the primary plate reveal β-III-tubulin-positive processes emanating from the colonies (FIG. 1C). Similar structures are found in SKC-infected tail tip fibroblast cells transfected with Oct4-encoding mRNA (FIG. 2). The colonies are picked 18 days post-infection and plated in NS propagation medium. NSC-like cells grow out of the spheres (FIG. 1D) and can be kept in adherent culture (FIG. 1E).

### Example 2

### Isolation of INS Clones

After 18-22 days neurosphere-like colonies are picked, transferred to Polyornithine/Laminin (POL)-coated culture dishes and kept in NS propagation medium (Euromed-N, EuroClone, Siziano, Italy), 1× N2 (Life technologies, Carlsbad, Calif.), 10 ng/ml bFGF (Life technologies, Carlsbad, Calif.) and 10 ng/ml EGF (R&D Systems, Minneapolis, Minn.). During the first 24 hours neurosphere-like colonies attached to the culture dish and iNS cells start to migrate out. 2 days later cells are washed with PBS (Life technologies, Carlsbad, Calif.) and dissociated for 5 min with 0.5% Trypsin/EDTA solution (Life technologies, Carlsbad, Calif.) to achieve a single cell suspension. Trypsin activity is inhibited with Trypsin Inhibitor (Life technologies, Carlsbad, Calif.). For maintenance culture 7×10⁵ cells are seeded onto POL-coated 6-cm culture dishes and splitted every 3 to 4 days. The medium is changed every other day, while EGF and bFGF are applied daily.

iNSCs are characterized by RT-PCR analyses, immunofluorescence stainings as well as microarray analyses:

Immunofluorescence Analyses

Cells are fixed in paraformaldehyde solution (4% in PBS; Carl Roth, Karlsruhe, Germany) for 15 minutes at room temperature and incubated in blocking solution (5% fetal bovine serum (Life technologies, Carlsbad, Calif.) in PBS with or without 0.1% Triton X-100 (Sigma, Saint Louis, Mo.) for 1 h. Afterwards, the cells are treated overnight with antibodies to Nestin (ms IgG, 1:100: Millipore, Billerica, Mass.), Pax6 (rb IgG, 1:100, Covance, Princeton, N.J.), Olig2 (rb IgG, 1:700, Chemicon/Millipore, Billerica, Mass.), Sox2 (ms IgG; 1:500; Systems, Minneapolis, Minn.), BLBP (rb IgG; 1:100; Abcam, Cambridge, Mass.), Vimentin (ms IgG; 1:100; Chemicon/Millipore, Billerica, Mass.), TUJ1 (ms IgG, 1:1000; Covance, Princeton, N.J.), GFAP (rb IgG; 1:1000; DAKO, Hamburg, Germany), MAP2ab (ms IgG; 1:700; Sigma, Saint Louis, Mo.), GABA (rb IgG; 1:500; Sigma, Saint Louis, Mo.), and Synapsin 1 (ms IgG; 1:500; Synaptic Systems, GOttingen, Germany). Secondary Alexa488- or Alexa555-labeled antibodies (1:1000; Life technologies, Carlsbad, Calif.) are used to detect and visualize the primary antibodies. All antibodies are diluted in blocking solution. Micrographs are taken with an Axiovert 200M microscope (Carl Zeiss, Jena, Germany).

Reverse Transcription Polymerase Chain Reaction (RT-PCR)

mRNA is isolated from cultured cells using the RNAeasy kit (Quiagen, Hilden, Germany) according to the manufacturer's recommendations. The reverse transcription is carried out with the iScript cDNA synthesis kit (Bio-Rad, Berkeley, Calif.). The RT-PCR analyses for the detection of neural stem cell specific mRNAs are performed as described in Glaser et al. (2007, PLoS One 2, e298). RT-PCR analyses for the detection of transgenic Klf4, Sox2 and c-myc are carried out using the primer pairs listed in table 1 and FIG. 3. PCR-program: 95° C. 2 min, 95° C. 30 sec, X° C. 30 sec, 72° C. 1 min, 72° C. 10 min. Steps 2- 4 are repeated 35 times.

TABLE 1

| Target | forward primer | reverse primer | annealing Temp. |
|---|---|---|---|
| KIf4TG | AGGCACTACCGCAAACACAC | TTTATCGTCGACCACTGTGC | 60° C. |
| Sox2TG | GCCCAGTAGACTGCACATGG | CCCCCTTTTTCTGGAGACTA | 60° C. |
| c-MycTG | CAGAGGAGGAACGAGCTGAAGCGC | TTTGTACAAGAAAGCTGGGT | 60° C. |
| Oct4 | TCTTTCCACCAGGCCCCCGGCTC | TGCGGGCGGACATGGGGAGATCC | 60° C. |
| Sox2 | TAGAGCTAGACTCCGGGCGAT | TTGCCTTAAACAAGACCACGAAA | 60° C. |
| Nanog | CAGGTGTTTGAGGGTAGCTC | CGGTTCATCATGGTACAGTC | 60° C. |

Micoarray Data Analysis

RNA is isolated using the RNeasy-Kit (Qiagen, Hilden, Germany). Expression analysis is performed following the Illumina (Illumina Inc., San Diego, Calif., USA) Whole-Genome Gene Expression Direct Hybridization Assay analysis pipeline. mRNA transcription levels are evaluated using the MouseWG-6 (version 2, revision 3) array which queries 45281 probes in 30854 genes and described mRNA features. All samples are processed in at least triplicates to reduce signals arising from processing artifacts. Data processing is performed using the GenomeStudio suite version 2011.1 and the Gene Expression module version 1.9.0 (both Illumina Inc., San Diego, Calif., USA). Differential analysis is performed against the MEF fibroblast intensities applying the average normalization algorithm. The Man-Whitney error model and the correction for multiple testing by Benjamini-Hochberg false discovery method is used. Filtering excludes all probes with a detection p-value of greater than 0.01.

### Example 3

### Investigation of Artificially Induced iNSCs

To investigate the identity of the induced NSCs (iNSCs) comprehensive molecular characterization at the protein and mRNA level is performed.

Immunostaining reveals that the transdifferentiated cells consistently express numerous neural stem cell markers including Nestin and Olig2 (FIGS. 1F,G), Sox2 and Blbp (FIG. 1H). RT-PCR analysis of transdifferentiated cells confirm transcription of Pax6, Blbp and Sox2 (FIG. 2), all of which are characteristic markers of NSCs (Conti et al. 2005, PLoS Biol 3, e283).

The capability of iNSCs to self renew and grow clonally under proliferation conditions is also assessed. The derivation of iNSCs is reproduced in three independent experiments each employing 130,000 cells and yielding between 7 and 11 neurosphere-like structures. In total, five of these structures are isolated and four of them can be stably expanded. All four iNS lines analyzed express NSC markers and are expandable for more than 50 passages without changing their morphology and growth properties (FIGS. 1J,K). The mean doubling times of iNS lines are found to be similar to that of brain-derived control NSCs (FIG. 1P). The characteristic profile of NSC marker expression does not change after prolonged passaging as judged by staining for Pax6 and Nestin (FIGS. 1L,M).

To assess the secondary neurosphere-forming potential of iNSCs single cell suspensions are generated that are cultured in non-coated flasks forming secondary neurospheres (FIG. 1N) that are able to grow again in adherent culture (FIG. 1O). Using this approach, more than 60% of the spheres can be expanded. In conclusion, fibroblast-derived transdifferentiated cells represent a homogenous proliferating cell population resembling a radial glia NSC type (Conti et al. 2005, PLoS Biol 3, e283).

### Example 4

### Analysis of the Similarities Between iNSCs and Control NSCs

To analyze the similarities between iNSCs and control NSCs comparative global gene expression data by microarray analysis are generated.

Scatter plots of the scores for MEFs and ES cells, respectively, versus iNS-2 cells (FIGS. 4A,B) reveal a significant difference between iNSCs and their origin MEFs, as well as pluripotent cells. In contrast, iNS-2 cells are very similar to the control NSC line (FIG. 4C) and the sister clone iNS-5 isolated from the same transdifferentiation experiment (FIG. 4D). Hierarchical cluster analysis reveals a high degree of similarity between all NS lines independent of their origin (FIG. 4E). Although there are subtle differences in the global gene expression profiles of the three iNS lines tested all of them are clearly distinct from both, MEFs and ES cells. One of the established iNSC lines (iNS-2) is particularly similar to the NS control line derived from fetal brain (FIGS. 4C,E,F). Numerous genes known to be involved in NSC self renewal or neural determination, such as Foxg1, Nes, Bmi1, and Olig2 are strongly upregulated in iNSCs and NSCs compared to MEF cells (FIG. 4F). Transcription of several fibroblast-specific genes such as Col1a1, Col3a1, Dkk3 and Thy1 is downregulated (FIG. 4F), but there is also a subset of genes exhibiting similar expression in MEF and iNSCs (FIG. 3A) suggesting some residual fibroblast epigenetic memory.

In order to examine whether iNSCs exhibit a regional identity region-specific transcription factors are selected from the microarray data set and assessed via RT-PCR analysis. Although both, iNSCs and NSC controls, share high expression of some forebrain markers such as Emx2, Foxg1, and Nr2e1 they exhibit low expression of forebrain-specific Emx1 (FIGS. 3B,C). Some mid/hindbrain-specific mRNAs including Gbx2 and Egr2 are highly abundant in contrast to Pax2 that exhibits low transcript levels. Ventral markers Olig2 and Nkx2.2 are found highly expressed and low expression of dorsal markers Pax3 and Pax7. In conclusion, like their counterparts derived from brain, iNSCs do not fully correlate with a specific regional identity but are mostly compatible with a ventral fore/mid/hindbrain fate.

RT-PCR analyses further reveals that none of the four iNSC lines analyzed expresses transgenic Sox2, Klf4 or c-Myc. However, all of the lines exhibit strong induction of endogenous Sox2 expression and no expression of endogenous Oct4 (FIG. 4G). To verify the initial requirement for the transdifferentiation factors genomic PCR analysis are performed revealing that the four iNS lines analyzed carry genomic integrations of the all three reprogramming transgenes (FIG. 4H).

### Example 5

### Differentiation of iNSCs into Neurons, Astrocytes and Oligodendrocytes In Vitro

The developmental potential of iNSCs is examined by assessing their capacity for differentiation into the three main neural lineages.

iNSC Differentiation

For the generation of astrocytes iNSCs are kept in 10% FCS-containing medium supplemented with 1× NEAA and 2 mM L-glutamine. For neuronal differentiation iNSCs are plated onto POL-coated dishes and kept in a 1:1 mix of Neurobasal Medium and DMEM/F12 supplemented with N2, B27 and 10 ng/ml BDNF, as well as 200 μM ascorbic acid (Sigma, Saint Louis, Mo.). Half of the medium is replaced every other day. After 2 weeks of culture the ratio is changed to 3:1 and N2 supplement reduced to 0.5%. To derive oligondendrocytes, iNSCs are cultivated in DMEM/F12 with 1× N2, 10 ng/ml PDGF (R&D Systems, Minneapolis, Minn.) and 10 μM Forskolin (Sigma, Saint Louis, Mo.) for 4 days. Afterwards, PDGF and Forskolin are replaced by 30 ng/ml 3,3,5-triiodothyronine (T3) hormone and 200 μM ascorbic acid (all from Sigma, Saint Louis, Mo.) for another 7 days.

Results

Astrocytic differentiation of iNSCs is induced by exposure to FCS, yielding cells with astrocyte morphology that uniformly stain for glial fibrillary acidic protein (GFAP) (FIG. 5A). To target differentiation of iNSCs towards the oligodendroglial fate a protocol employing media containing forskolin, triiodothyronine and ascorbic acid is used. Staining reveals oligodendrocyte marker O4-positive cells with characteristic morphology (FIG. 5B). For neuronal differentiation iNSCs are cultivated in the absence of EGF/FGF but presence of BDNF. Differentiated cells show neuronal morphology and expression of the neuronal markers βIII-tubulin (FIG. 5C) and NeuN (FIGS. 5D,E) as well as microtubule-associated protein (MAP2) (FIGS. 5C,F). Quantification of the differentiated cells demonstrates that the differentiation potential of iNSCs is high for astrocytes and neurons whereas oligodendroglial differentiation is relatively rare (FIG. 5G). Thus, iNSCs are very similar to their counterparts derived from brain tissue for which oligodendrocyte differentiation is challenging (FIG. 6A) (Conti et al. 2005, PLoS Biol 3, e283). Nevertheless, the differentiation spectrum of iNSCs is not restricted to neurons and astrocytes but extends also to oligodendrocytes. All three iNS lines analyzed exhibit a similar potential to differentiate into neurons (FIG. 6B). Further cellular characterization of iNS-derived neurons reveals that the majority developed a GABAergic phenotype (FIG. 5H) and are able express synaptic proteins (FIG. 5I).

To analyze whether the resulting neurons exhibit functional membrane properties whole-cell patch-clamp recordings after 3 weeks of differentiation are performed.

Patch Clamp Analysis For patch-clamp measurements cells cultured on plastic coverslips (Nunc, Roskilde, Denmark) are transferred to a chamber that is mounted to an x-y stage and continuously super- fused with artificial cerebrospinal fluid (aCSF; (in mM): 140 NaCl, 3 KCl, 2 CaCl2, 1 MgCl2, 25 D-glucose, and 10 HEPES/NaOH (pH 7.35, 305-315 mosmol/kg) at 2 ml/minutes. Recordings are performed at room temperature. Cells are visualized using an upright microscope equipped with near-infrared differential interference contrast and x 60 water immersion objective (Nikon). Whole-cell current-clamp and voltage-clamp recordings are carried out with an Axopatch-200B amplifier (Axon Instruments) that is interfaced by an ND-converter (Digidata 1320; Axon Instruments,) to a PC running PClamp software (version 9; Axon Instruments). The patch pipette (tip resistance 3-5 MΩ) contains the following (in mM): 120 potassium gluconate (C6H11O7K), 20 KCl, 10 NaCl, 10 EGTA, 1 CaCl2, 4 Mg ATP, and 0.4 Na GTP, 10 HEPES/KOH (pH 7.2, 290 mosmol/kg). Local application of either 15 mM L-glutamate or 15 mM GABA is used to assess the expression of functional neurotransmitter receptors.

Results

This analysis reveals the expression of a complex outward current pattern including inactivating and sustained components (FIG. 5J). This pattern is reminiscent of neurons expressing both A-type and delayed rectifier potassium channels. In contrast to these high-amplitude outward currents, transient inward currents are only of small amplitude. However, in current-clamp experiments some cells (5 of 7) are able to fire an action potential, demonstrating membrane excitability (FIG. 5K). Furthermore, the neurons display surface expression of glutamate and GABA_{A} receptors (n=5 and n=3, respectively, FIGS. 5L,M), which is a prerequisite for the formation of glutamatergic and GABAergic synapses.

### Example 6

### Differentiation of iNSCs into Neurons, Astrocytes and Oligodendrocytes In Vivo

Studies to assess the in vivo developmental potential of iNSCs are also conducted, especially concerning their suitibility for glial cell replacement.

Transplantation of iNS Cells

Cells are transplanted into early postnatal (P1) myelin deficient rats (md rats) as described in Glaser et al. 2007, PLoS One 2, e298: Cells are detached using trypsin/EDTA solution and concentrated in PBS/0.1% BSA to 125,000 cells/ul. Pups are anesthetized by short hypothermia on crushed ice. 5 μl of cell suspension are transplanted into the left and right hemispheres (three injection sites per hemisphere with a total volume of 6 μl per animal) through a pulled glass capillary. 14 days post-transplantation the recipients are anesthetized (10 mg/kg Xylazine, 80 mg/kg Ketanest) and transcardially perfused with 4% paraformaldehyde in PBS. The brains are dissected, postfixed with 4% paraformaldehyde in PBS at 4° C. over night and kryoprotected in 30% sucrose in PBS. Brains are embedded in Tissue-Tek (Sakura, Alphen aan den Rijn, Netherlands) and frozen at −70° C. for at least 1 h. 40 μm cryosections are made. Brain slices are washed with PBS, treated with 100% ethanol for 10 minutes and incubated in blocking solution for 2 h (5% BSA (Carl Roth, Karlsruhe, Germany), 0.1% Triton X (Sigma, Saint Louis, Mo.) in PBS). For the immunofluorescence analyses, slices are incubated with primary antibodies targeted against oligodendroglia proteolipid protein (PLP) (rb; 1:100, Abcam, Cambridge, UK), which is deficient in the and rat brain, the murine neural marker M2 (rat; 1:250, Developmental Studies Hybridoma Bank, Iowa City, Iowa), NeuN. (ms; 1:50, Millipore, Billerica, Mass.) and GFAP (rb; 1:1000; DAKO, Hamburg, Germany) overnight at room temperature. To detect the primary antibodies, slices are incubated with Alexa488-, Cy3- and Cy5-conjugated secondary antibodies for 4 h at room temperature. Pictures are taken using a Zeiss Apoptome microscope and an axiovison camera (Carl Zeiss, Jena, Germany)

Results

These in vivo studies reveal iNS-derived M2-positive cells with astrocyte morphology in a variety of host brain regions including cortex and striatum (FIG. 7). Many of the M2-positive profiles can be double-labeled with an antibody to GFAP (FIGS. 7B,C). In addition, PLP-positive profiles are detected in white matter structures such as the corpus callosum (FIG. 7D). These data clearly demonstrate that grafted iNSCs survive and give rise to differentiated neural cells in vivo.

1. A method of deriving an induced neural stem cell (iNCS) by nuclear reprogramming of a somatic cell, wherein the method comprises a step of contacting the somatic cell with an Oct4 protein or a functionally equivalent analogue, variant or fragment thereof for a limited time period.

2. The method according to claim 1, characterized in that the limited time period is 10 days or less.

3. A The method according to claim 1, characterized in that the step of contacting the somatic cell with the Oct4 protein or a functionally equivalent analogue, variant or fragment thereof is accomplished by delivery of cell-permeable Oct4 protein or its functionally equivalent analogue, variant or fragment, or by delivery of mRNA encoding Oct4 protein or its functionally equivalent analogue, variant or fragment, or by infection employing non-integrating viruses, or by doxycycline- induced expression of Oct4 protein or its functionally equivalent analogue, variant or fragment.

4. The method according to claim 1, characterized in that the somatic cell is a human cell.

5. The method according to claim 1, characterized in that the somatic cell is a fibroblast or a keratinocyte.

6. The method according to claim 1, characterized in that the induced neural stem cell is derived directly from the somatic cell, without passing through a pluripotent stage.

7. The method according to claim 1, characterized in that the method further comprises a step of contacting the somatic cell with one or more transcription factors selected from Sox2, cMyc and Klf4 and their functionally equivalent analogues, variants and fragments.

8. The method according to claim 7, characterized in that the somatic cell is contacted constitutively with the transcription factors Sox2, cMyc and Klf4 or their functionally equivalent analogues, variants or fragments.

9. The method according to claim 1, characterized in that the somatic cell is contacted with one, two, three, or more additional neurogenic transcription factors.

10. The method according to claim 1, characterized in that the induced neural stem cell further differentiates into a neuron, an astrocyte or an oligodendrocyte.

11. The method according to claim 1, characterized in that the method is conducted in vivo or in vitro.

12. An induced neural stem cell obtained by a method according to claim 1.

13. A neuron, an astrocyte or an oligodendrocyte obtained from an induced neural stem cell according to claim 12.

14. The method, according to claim 2, wherein the limited period of time is 3 to 5 days.

15. The method, according to claim 9, wherein the additional neurogenic transcription factor is selected from TLX, Bmi1, Hes5, Brn1, Brn2, Brn4, Pax6, Sox1, Sox3, Sox10, PLZF, Hes1, Dach1, Dlx1, Gli1, Gli2, Gli3, ID2, ID4, Olig2 and their functionally equivalent analogues, variants and fragments.
